(19) United States
(12) Patent Application Publication
(10) Pub. No.: US 2019/0117129 A1
(43) Pub. Date: Apr. 25, 2019

(54) SYSTEMS, DEVICES, AND METHODS FOR DETERMINING AN OVERALL STRENGTH ENVELOPE

(71) Applicant: Arizona Board of Regents on Behalf of the University of Arizona, Tucson, AZ (US)

(72) Inventor: Marvin J. Slepian, Tucson, AZ (US)

(73) Assignee: Arizona Board of Regents on Behalf of the University of Arizona, Tucson, AZ (US)

(21) Appl. No.: 16/309,792

(22) PCT Filed: Jun. 16, 2017
(86) PCT No.: PCT/US17/37898

§ 371 Date: Dec. 13, 2018

Related U.S. Application Data
(60) Provisional application No. 62/351,258, filed on Jun. 16, 2016.

(51) Int. Cl. A61B 5/11; A61B 5/22; A61B 5/00; A61B 5/0205; A61B 5/145

(52) U.S. Cl. CPC A61B 5/1121; A61B 5/224; A61B 5/1114; A61B 5/1128; A61B 5/6801; A61B 5/6867; A61B 5/02405; A61B 5/14546; A61B 5/4266; A61B 5/7425; A61B 5/486; A61B 5/0022; A61B 5/0205

(57) ABSTRACT

Systems, methods, and software products determine an overall motion, flexibility and/or strength envelope for an individual. Force, movement and position data of the individual is captured and processed to generate an overall motion, flexibility and/or strength envelope for the individual that defines overall motion, flexibility and/or strength of the individual. The system includes a flexibility server with memory and a processor that are adapted to receive force, movement and position data of the individual. A motion, flexibility and/or strength analyzer of the system, implemented as machine readable instructions stored in the memory and executed by the digital processor, is capable of processing the force, movement and position data to generate an overall motion, flexibility and/or strength envelope for the individual that defines the overall motion, flexibility and/or strength of the individual.

### RELATED APPLICATIONS

This application claims priority to U.S. Patent Application Ser. No. 62/351,258, titled “Systems, Devices, and Methods for Determining an Overall Strength Envelope”, filed Jun. 16, 2016, and incorporated herein by reference.

### BACKGROUND

Movement is a critical activity and state for all organisms, including man. Movement may be considered as being of two types: complete translation or translocation of an organism from one location to another—examples here being walking, running, somersaulting, falling or the like; or alternatively movement may be considered the displacement, bending, angulation, rotation, or other positional alteration of the organism or components of the organism—e.g. a limb, the head and neck, the upper torso, the trunk without significant translocation of the overall organism as in going from location A to location B. Although strength of particular joints may be quantified, there is no measurement or quantification of overall strength of an organism.

### SUMMARY

The embodiments disclosed herein are focused on translocation of an organism from one location to another. As such considering this type of movement, an organism, such as a human or animal has the ability to move all or a portion of its body, soma or corpus. Movement of the body, body part or appendage may be either active—e.g. as in bending over, lifting an arm, tilting the head and the like, actuated via internal neural commands and intention, or passive. For passive movement another individual, device or system moves the appendage such as in someone moving a test subjects arm to determine what has been colloquially referred to as range of motion, but is more accurately a “range of flexibility.” Within both of these forms or movement there is a defined maximum range of movement that is three-dimensional. If one were to trace the outer perimeter and capture and visualize the entire “volume” of space covered by this movement this would be define, both quantitatively and qualitatively, the “motion or movement envelope”—i.e. for active, volitional or stimulated movement or the “flexibility envelope” for either active or passive movement of the body region, appendage, limb, head and neck, and the like being tested.

For the embodiments disclosed herein, “motion envelope” and “movement envelope” may be utilized interchangeably.

As to the “flexibility envelope,” this is a region that may be equal to, larger or smaller than the motion or movement envelope. The size, volume or extent of the flexibility envelope is governed by the limberness, elasticity, laxity and otherwise overall flexible nature of the body region, appendage or limb being tested. The degree of flexibility of a given organism or test subject varies depending upon how much activity the organism regularly performs, the degree of intactness, i.e. freedom from injury, and on the age or state of health of the organism. The extent of the flexibility envelope is also governed by whether the activity performed to define the envelope is active and volitional or passive by a third party or system.

It should be understood that the motion and flexibility envelope may overlap in full or in part. For example, an individual may only be able to move a limb actively to a defined limit or cover a defined volume of space. However, with an assistant moving the limb, the range of motion may be greater, largely governed by the “flexibility” of the limb. In this case the passive “flexibility envelope” may be slightly greater than the active “motion envelope.” In contrast, an individual may be able to actively move a limb to a defined degree and as such the “flexibility” and “mobility” envelopes will overlap. It is envisioned that with the embodiments disclosed herein, both the motion, flexibility and/or strength envelopes may be defined and their degree of overlap or non-overlap determined.

For humans, motion, flexibility, and strength are typically measured individually as a single parameter for a single appendage. For example, when a person has an injury that affects an elbow joint, such as a broken arm that requires immobilization of the elbow joint to allow the bone to heal, this immobilization typically results in reduced flexibility and strength of the elbow joint. Rehabilitation of the joint typically requires physiotherapy to improve range of motion and flexibility of the elbow joint, where motion, flexibility and/or strength is measured in angular degrees of movement of the forearm relative to the upper arm. Despite this characterization of the movement, much information such as the full geometric excursion of the contiguous or involved tissue, appendage or limb, is not provided by this technique.

The embodiments disclosed herein, defining, quantitating, analyzing and otherwise utilizing the motion and flexibility envelope, addresses this limitation and provides novel means of completely capturing movement and flexibility information, both quantitatively and qualitatively, analyzing, storing, displaying, comparing, telemetering and otherwise utilizing this data.

In one embodiment, a method determines an overall motion, flexibility and/or strength envelope for an individual. Force, movement and position data of the individual is captured and processed to generate an overall motion, flexibility and/or strength envelope for the individual that defines overall motion, flexibility and/or strength of the individual.

In another embodiment, a system determines an overall motion, flexibility and/or strength envelope for an individual. The system includes a flexibility server having memory and a processor and adapted to receive force, movement and position data of the individual, and a motion, flexibility and/or strength analyzer, implemented as machine readable instructions stored in the memory and executed by the digital processor. The motion, flexibility and/or strength analyzer is capable of processing the force, movement and position data to generate an overall motion, flexibility and/or strength envelope for the individual defining overall motion, flexibility and/or strength of the individual.

In another embodiment, a system determines an overall motion envelope for an individual, including means for capturing force and motion of the individual, means for recording and analyzing said force and motion data, means for storing raw and processed information, means for converting the force and motion data into a motion, flexibility and/or strength envelope, means for displaying the motion, flexibility and/or strength envelope, and means for comparing the motion, flexibility and/or strength envelope at differing time points and under differing conditions.

In another embodiment, a method determines an overall motion, flexibility and/or strength envelope for an individual. Force, movement and position data is captured from at least one sensor or motion detection means configured with the individual. The force, movement and position data is analyzed to generate the overall motion, flexibility and/or strength envelope for the individual and a display from the overall motion, flexibility and/or strength envelope is generated to show motion, flexibility and/or strength of the individual.

In another embodiment, a software product has instructions, stored on non-transitory computer-readable media, wherein the instructions, when executed by a computer, perform steps for determining an overall motion, flexibility and/or strength envelope for an individual. The software product includes instructions for capturing force and movement data, instructions for analyzing the force and movement data, instructions for converting the force and movement data into a graphical representation of movement in either 1, 2, 3 or 4D, and instructions for comparing movement of any aspect of full or partial motion, flexibility and/or strength envelope to either another point of time of the envelope of the same individual or of a database of performance.

### BRIEF DESCRIPTION OF THE FIGURES

FIG. 1 is a 2-D high level visualization of one example overall motion, flexibility and/or strength envelope for an individual human body, in an embodiment.

FIG. 2A shows example motion, flexibility and/or strength components that form an overall motion, flexibility and/or strength envelope of the individual, in an embodiment.

FIG. 2B shows an example grouping of external and internal components to form the internal/external group of FIG. 2A.

FIG. 3 shows one example system for determining the overall motion, flexibility and/or strength envelope of FIGS. 1 and 2 for an individual, in an embodiment.

FIGS. 4 and 5 show a right side view and a front view of the individual of FIG. 3 illustrating example positioning of sensors, in an embodiment.

FIG. 6 shows one example system that utilizes the flex server of FIG. 3 together with a plurality of cameras mounted to capture images within a room to determine the overall motion, flexibility and/or strength envelop of the individual, in an embodiment.

FIGS. 7, 8 and 9 show example movement of the left-shoulder of the individual.

FIG. 10 shows one example rendering of shoulder motion, flexibility and/or strength as generated from the overall motion, flexibility and/or strength envelope of FIGS. 12, 3, and 6, in an embodiment.

FIG. 11 shows the motion, flexibility and/or strength envelope of FIG. 1 and an exemplary overlapping comfort/discomfort envelope, in an embodiment.

### DETAILED DESCRIPTION OF THE EMBODIMENTS

The embodiments disclosed herein describe systems, devices, and methods for evaluating overall movement and flexibility of an organism (e.g., the human body), of collecting quantitative information and statistics on overall movement and flexibility for different types of organisms, and for evaluating an individual organism's movement and flexibility either standalone or against changes from other interval recording of the same or against the statistical information based upon the type of organism. An organism's current overall motion, flexibility and/or strength envelope is evaluated against a recorded overall motion, flexibility and/or strength envelope for that individual organism or against a large database (of continuously updated—i.e. akin to machine learning) to determine trends in motion, flexibility and/or strength over time. This overall motion, flexibility and/or strength envelope defines motion, flexibility and/or strength of the organism, where individual portions of the overall motion, flexibility and/or strength envelope may be measured and defined in many various ways, such as x-y-z plane movements, pitch-roll-yaw movements, polar or circular coordinates, and so on. In addition to dimensional data (1, 2, 3 or 4)(time)D, velocity data (e.g. dx/dt), or acceleration data (dv/dt) for any point, plane or space may be characterized as well. However determined, the individual motion, flexibility and/or strength measurements are combined to form the overall motion, flexibility and/or strength envelope that is stored digitally and may be processed, evaluated, and presented in many recognizable and usable forms to indicate overall motion, flexibility and/or strength of the organism or of components of the organism—e.g., a given appendage such as an arm.

In the following examples, the human body is used to illustrate the embodiments hereof, however, the systems, devices, and methods described herein may apply to any organism where overall motion, flexibility and/or strength of movement may be measured without departing from the scope hereof.

A human body has two-hundred and thirty movable joints, each with a certain range of motion based upon the type of joint. The human body has three types of joint: (a) fibrous joints that are held together by strong connective tissue with only a slight capacity to stretch and have very little movement between the joined bones, providing great stability; (b) cartilaginous joints that allow for slight movement and occur where bone ends are covered by a somewhat flexible, compressible connective tissue called cartilage; and (c) synovial joints that consist of a capsule of connective tissue that encloses a space or cavity between the bones to allow the greatest degree of movement.

The body also has approximately 700 muscles, each with a certain range of motion based upon the location thereof, where each movement has an associated strength. The human body has three types of muscles: (a) visceral muscles found inside of organs like the stomach, intestines and blood vessels controlled by the unconscious part of the brain; (b) cardiac muscles found in the heart responsible for pumping blood throughout the body; and (c) skeletal muscles which are voluntary muscle tissue controlled consciously that control movement of various parts in the body.

Measuring a range of motion for a specific joint or muscle on a body does not provide a complete indication of overall motion, flexibility and/or strength of that body. Certain embodiments disclosed herein provide quantification of overall motion, flexibility and/or strength and an overall motion, flexibility and/or strength envelope for any organism.

In addition to distinct joints—which may be conceptualized as hinges, defined body elements—e.g. appendages (arms/legs), the head and neck and the trunk, the upper torso and the lower torso may be envisioned as “flex elements.” A flex element of a corpus is herein defined as any element of the corpus of an organism—either contiguous or discontinuous (not immediately adjacent), that regardless of physical proximity are considered as a functional group. Flex elements may contain internal and external structures as well. The described embodiments provide methods, devices and systems to measure, codify, quantitate, analyze, store, telemeter and compare flex elements as well.

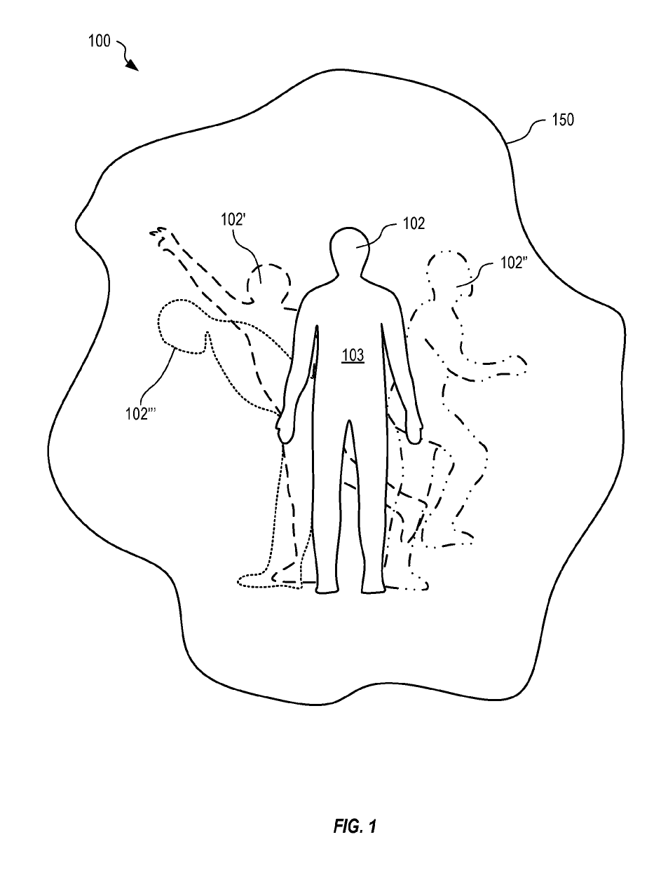

FIG. 1 is a 2-D high level visualization of an overall motion, flexibility and/or strength envelope 150 for an individual human body 102 (hereinafter individual 102). It should be understood that the full or max flex envelope is actually a 3-D structure at a given moment of time and a 4D structure taking time into account. The overall motion, flexibility and/or strength envelope 150 encompasses and defines all possible non-traumatic/accidental movements of individual 102, whether active (i.e. performed by the test subject themselves or passive (i.e., performed by an exogenous testing individual, device or system). For example, as individual 102 participates in various activities (e.g., stretching individual 102′, running individual 102″, and bending individual 102′″), overall motion, flexibility and/or strength envelope 150 expands to encompass the demonstrated motion, flexibility and/or strength of individual to perform those activities.

Overall motion, flexibility and/or strength envelope 150 changes for individual 102, for example as individual 102 ages from birth through childhood, overall motion, flexibility and/or strength envelope 150 expands to encompass the increased motion, flexibility and/or strength of the child as he/she becomes mobile. Where individual 102 participates in advanced training (e.g., Yoga, sporting activities, weight lifting and so on), overall motion, flexibility and/or strength envelope 150 increases as a result of the body 103 becoming more flexible, limber and stronger through the training. As individual 102 ages through senior years towards the end of his/her life cycle, overall motion, flexibility and/or strength envelope 150 decreases as the body 103 loses flexibility and strength through decline and decay. Where individual 102 changes body rhythms, such as patterns in regular activity, overall motion, flexibility and/or strength envelope 150 also changes. For example, where individual 102 has been exercising regularly twice a week for many months, overall motion, flexibility and/or strength envelope 150 encompasses the resulting flexibility and strength achieved by such exercise. However, if individual 102 then stops exercising regularly, overall motion, flexibility and/or strength envelope 150 reduces as individual 102 loses flexibility and strength due to reduced activity. In another example, individual 102 suffers an injury and overall motion, flexibility and/or strength envelope 150 is reduced. As individual 102 recovers from the injury, and possibly uses appropriate rehabilitation, overall motion, flexibility and/or strength envelope 150 increases again. By comparing a current overall motion, flexibility and/or strength envelope 150 for individual 102 with a previously recorded overall motion, flexibility and/or strength envelope 150, changes in overall motion, flexibility and/or strength can be determined and visualized, thereby allowing individual 102 (or a medical practitioner) to better understand the effects of rehabilitation.

Overall motion, flexibility and/or strength envelope 150 is also affected in the short term. For example, where individual 102 has been confined to an aircraft seat as a passenger, upon disembarking from the aircraft, overall motion, flexibility and/or strength envelope 150 of individual 102 is temporarily reduced. Similarly, after participating in a sporting activity, overall motion, flexibility and/or strength envelope 150 of individual 102 may be temporarily extended because of warm and stretched muscles. Thus, overall motion, flexibility and/or strength envelope 150 is continually changing.

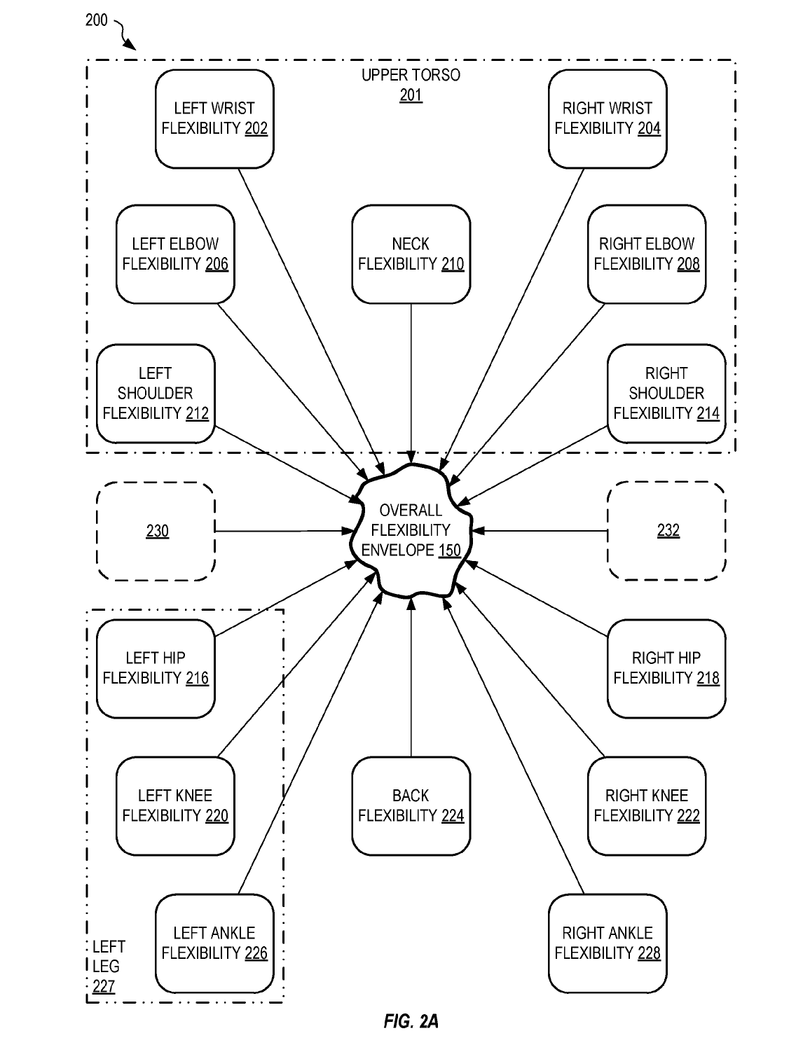

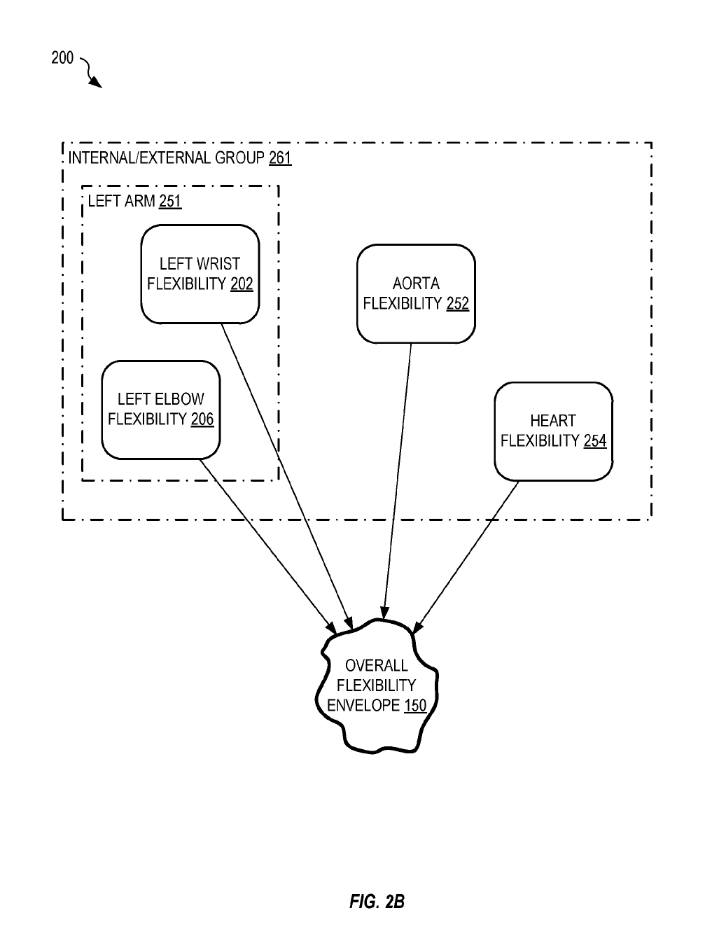

FIG. 2A shows example motion, flexibility and/or strength components that form overall motion, flexibility and/or strength envelope 150 of individual 102. Individual 102 has two-hundred and thirty movable joints and approximately seven hundred moveable muscles. Overall motion, flexibility and/or strength envelope 150 is based upon motion, flexibility and/or strength of at least some of these joints and muscles. Overall motion, flexibility and/or strength envelope 150 may be based upon motion, flexibility and/or strength of all of these joints and/or muscles; however, certain ranges of motion, and this flexibility and strength, may be difficult to measure. Therefore, in embodiments, motion, flexibility and strength of certain joints (e.g., the fibrous joint that limits movement between the tibia and fibula bones of the lower leg) and muscles (e.g. cardiac muscles) may be derived from indirect measurement.

FIG. 2A shows regional grouping of individual motion, flexibility and/or strength components (i.e., inputs 202-214) to form an upper torso 201 flexibility element. Similarly, components 216, 220, and 226 may be regionally grouped as a left leg 227 flexibility element. Other regions may be similarly grouped, where groups may be nested and overlapped.

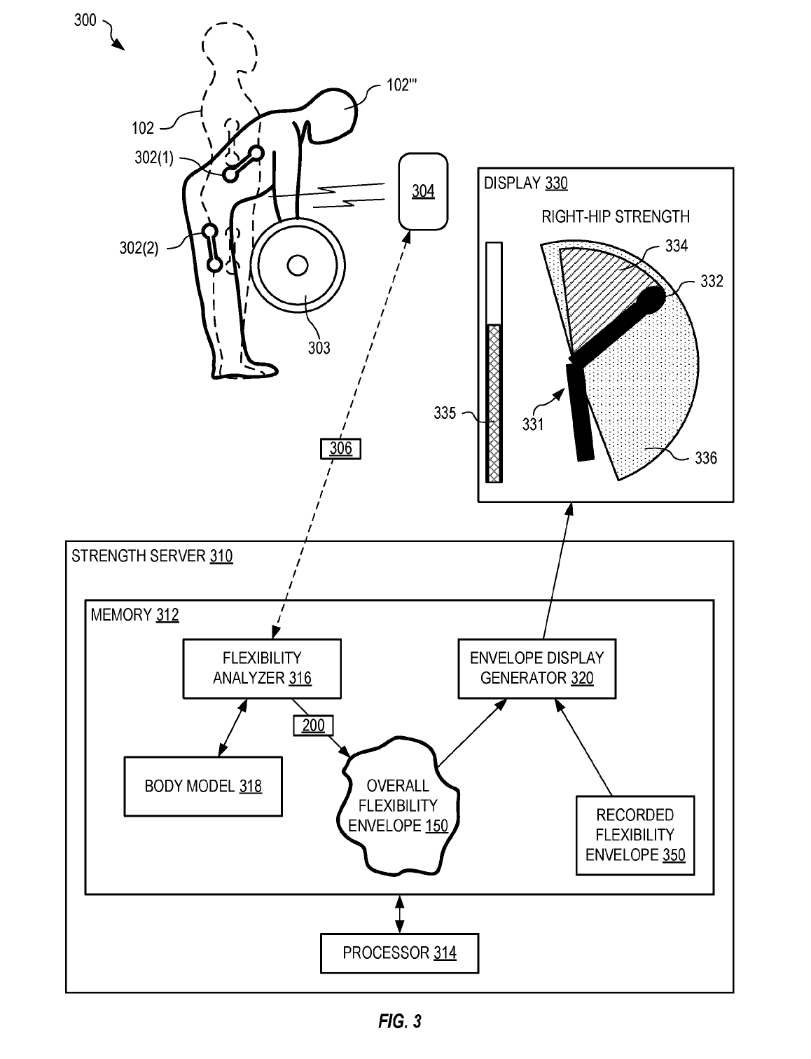

FIG. 2B shows example grouping of external and internal components to form an internal/external group 261. That is, external components of a left arm 251 flexibility element may be grouped with internal components such as aorta flexibility 252 and heart flexibility 254 to form internal/external group 261. In the case of internal components, such as aorta flexibility 252 and heart flexibility 254, one or more sensors may be implemented within the body of individual 102 and communicatively connected with a relay device (e.g., relay device 304 of FIG. 3) that collects and relays data wirelessly from these internal sensors.

In the example of FIGS. 2A and 2B, overall motion, flexibility and/or strength envelope 150 is illustratively shown derived from fourteen different motion, flexibility and/or strength inputs 200: left wrist motion, flexibility and/or strength 202, right wrist motion, flexibility and/or strength 204, left elbow motion, flexibility and/or strength 206, right elbow motion, flexibility and/or strength 208, neck motion, flexibility and/or strength 210, left shoulder motion, flexibility and/or strength 212, right shoulder motion, flexibility and/or strength 214, left hip motion, flexibility and/or strength 216, right hip motion, flexibility and/or strength 218, left knee motion, flexibility and/or strength 220, right knee motion, flexibility and/or strength 222, back motion, flexibility and/or strength 224, left ankle motion, flexibility and/or strength 226, and right ankle motion, flexibility and/or strength 228. Many of these motion, flexibility and/or strength inputs 200 are complex (i.e., combining multiple planes and ranges of motion). For clarity of illustration, FIG. 2 does not show all possible motion, flexibility and/or strength inputs 200. As noted above, the human body has two-hundred and thirty movable joints and about seven hundred muscles; thus, overall motion, flexibility and/or strength envelope 150 may be derived from many other motion, flexibility and/or strength inputs without departing from the scope hereof.

In certain embodiment, strength is directly associated with muscles that facilitate movement of the joint. Overall motion, flexibility and/or strength envelope 150 may be defined, in part, as motion in different planes of appendages of individual 102. Neck motion, flexibility and/or strength 210 may be derived from multiple measurements of motion, flexibility and/or strength. For example, individual 102 may be able to move his/her head backwards and forwards over a certain range with a certain strength in a first plane, sideways over a certain range with a certain strength in a second plane, and rotate his/her head through a certain range with a certain strength in a third plane. Strength and these ranges and planes combine to form neck motion, flexibility and/or strength 210.

Given the number of joints and muscles in the human body, this overall motion, flexibility and/or strength envelope is complex. For example, measurement of movement of a single joint or single muscle is only part of the overall motion, flexibility and/or strength envelope. Thus, in part, the overall motion, flexibility and/or strength envelope is a measurement of all motion, flexibility and/or strength of the body.

Capabilities and actions of individual 102 throughout the day depend upon the individual's motion capacity and flexibility. Thus, as a quantative measure, overall motion, flexibility and/or strength envelope 150 provides a signature, or status, of well-being of individual 102 at a particular moment.

However, overall motion, flexibility and/or strength envelope 150 is more than physical motion, flexibility and/or strength. It may also include emotional states that are based upon, or may influence, the physical mobility and flexibility of the individual. For example, where the individual is stressed, his/her muscles may be tense, resulting in less motion, flexibility and/or strength. Thus, overall motion, flexibility and/or strength envelope 150 may include other health information that relates to, or influences, the motion, flexibility and/or strength of the individual. That is, overall motion, flexibility and/or strength envelope 150 is derived based upon physical motion of one or more body components, appendage or flexibility elements (i.e., groups of components). The detected physical motion is governed by the material properties of the constituent component structures—i.e. muscle, ligaments, tendons, presence of edema, blood flow, venous and lymphatic drainage and the like. In addition to physical elements, hormonal, neural and emotional (e.g. stress, fear) factors and states also modulate this motion and degree of flexibility.

Overall motion, flexibility and/or strength envelope 150 is multi-dimensional and encompasses the entire body of individual 102. Thus, overall motion, flexibility and/or strength envelope 150 extends far beyond simple joint range of motion measurements (e.g., strength and movement of the elbow joint of one arm) that have a linear range—say 0-100, where 100 represents full range of motion and strength. Simple measurement of motion range and strength, also fails to take into account the three dimensional nature of many joint and muscle motions. Consider, for example, an individual has a broken arm that is set in a plaster cast. A simple evaluation of the individual's arm would indicate no range of motion or the lack of flexibility, but may indicate increased strength provided by the plaster cast. However, that simple assessment provides no indication of how the individual is coping with that injury. Instead, by measuring a current overall motion, flexibility and/or strength envelope 150 of individual 102 and comparing it to a previously recorded overall motion, flexibility and/or strength envelope of individual 102, differences in motion, flexibility and/or strength of other joints may indicate the further effect of the injury, and/or the plaster cast, on the entire body of individual 102.

Motion, flexibility and/or strength of individual 102 may be determined by detecting strength and movement of one or more body parts relative to one or more other body parts. For example, left elbow motion, flexibility and/or strength may be determined by detecting movement of a left forearm relative to a left upper arm. Such movement may be determined by one of three ways: implantable sensors that measure movement from within the body, wearable sensors that attach to the body to determine movement, and off the body sensing, where body movements is determined using external apparatus, such as in a wired room that utilizes one or more of machines and cameras to detect body movement. The movement (e.g., acceleration and speed of that movement) and knowing the mass (e.g., the individual's body mass or part thereof, or an object being moved) allows the strength to be estimated.

As outlined above, sensors may be implanted on, in or near body internal elements, organs or organ components. These sensors allow motion and flexibility determination of a defined “flex element.” For example, if one were to track the left upper extremity and the heart and lungs—these could be defined as a “flex element” or group.

FIG. 3 shows one example system 300 for determining overall motion, flexibility and/or strength envelope 150 for individual 102. System 300 includes a flex server 310 that has a non-transitory memory 312 and a digital processor 314. Flex server 310 is for example one or more computers enhanced to determine overall motion, flexibility and/or strength envelope 150 of individual 102.

In one embodiment, flex server 310 is configured to receive sensor data 306 from a plurality of movement sensors 302 that are attached to, or implanted within, individual 102. Movement sensors 302 may represent one or more of strain gauges, accelerometers, gyroscopes, displacement sensors, proximity sensors, hall effect sensors, optical encoders, potentiometers, linear and rotary sensors, eddy-current sensors, reflective light sensors, pressure sensors, force sensors, tilt sensors, vibration sensors, blood flow sensors, heart rate monitors, oxygen level sensors, and so on. Engineering has produced a plethora of sensors for all types of measurement and miniaturization of sensors and development of flexible circuitry allows movement sensors 302 to be implanted within the body of individual 102 or affixed on to the body of individual 102 such that continuous motion, flexibility and/or strength measurement is possible without disruption of activity of individual 102. See for example, www.MC10inc.com (e.g. the “Biostamp”). The embodiments hereof may use any type of sensor and any format as best suited to the measurements needed.

In the example of FIG. 3, a first movement sensor 302(1) is configured at a lower-back area of individual 102, and a second movement sensor 302(2) is configured at a right-thigh area of individual 102.

Sensors 302 are wirelessly coupled to a relay device 304 that relays sensor data 306, from sensors 302 to a flexibility analyzer 316 of flex server 310. In one embodiment, relay device 304 is a smartphone that uses Bluetooth to communicate with sensors 302 and a cellular network and/or Wi-Fi for communicating with flex server 310. Relay device 304 is carried by, or positioned proximate, individual 102 and periodically receives, time stamps, and stores data from sensors 302 within an internal memory. When within range and/or communicatively connected to flex server 310, relay device 304 sends sensor data 306 to flexibility analyzer 316. In one embodiment, each sensor 302 determines its own movement in three dimensions. In one embodiment, individual 102 inputs values for weight 303 being lifted or manipulated, such that strength analyzer 316 may determine strength in measured movement. System 300 may utilize other sensors and devices for measuring strength of individual 102 directly. For example, individual 102 may utilize exercise equipment the includes force sensors that measure forces applied by individual 102 to the exercise equipment.

Flexibility analyzer 316 has machine readable instructions stored within memory 312 that are executed by processor 314 to implement functionality for analyzing sensor data 306 and generating overall motion, flexibility and/or strength envelope 150. Flexibility analyzer 316 manipulates a body model 318 based upon sensor data 306 to determine motion, flexibility and/or strength inputs 200 for overall motion, flexibility and/or strength envelope 150. Body model 318 is configured to digitally model size, weight, and movement of individual 102. In one embodiment, body model 318 is configured with the location and type of movement sensors 302 configured with individual 102 and/or weight machines being used by individual 102. In one example of operation, flexibility analyzer 316 periodically receives sensor data 306 and uses sensor data 306 to manipulate body model 318 such that body model reflects and records movement and strength of individual 102. In one embodiment, body model 318 stores maximum movement ranges and determined strength for each joint of the human body based upon the input sensed positions and strength of individual 102. In another embodiment, body model 318 stores maximum movement ranges and determined strength for each muscle and joint of the human body based upon the input sensed positions of individual 102.

Flexibility analyzer 316 periodically retrieves motion, flexibility and/or strength ranges of each joint and/or muscle from body model 318 and sends these ranges as motion, flexibility and/or strength inputs 200 to overall motion, flexibility and/or strength envelope 150. Sensors 302, flexibility analyzer 316, and body model 318 cooperate to detect movement of individual 102 and to generate motion, flexibility and/or strength inputs 200 for input to overall motion, flexibility and/or strength envelope 150.

In one example of operation, sensor data 306 includes three dimensional position and/or movement information for each sensor 302. Flexibility analyzer 316 manipulates body model 318 based upon the received sensor data 306, such that body model 318 simulates movement of individual 102. For example, as shown in FIG. 3, as individual 102 bends at the waist, to the position shown as individual 102′″, body model 318 is manipulated to follow the movement indicated within sensor data 306 for sensors 302(1) and 302(2), the user's body weight, and weight 303 is also taken into account. It is noted that other parts of body model 318 also move to achieve the defined movement. For example, the shoulders, arms, and head have also moved relative to the right thigh (sensor 302(2)), although they remain positioned relatively stationary relative to the lower back (sensor 302(1)). Thus, by correctly modeling movements sensed by sensors 302(1) and (2), movement of other body parts and joints may be derived, even when sensors are not configured to directly measure those movements.

Flexibility analyzer 316 then reads flexibility and strength ranges (including derived flexibility and strength) of the right hip joint, from body model 318 and inputs right-hip motion, flexibility and/or strength 218 to overall motion, flexibility and/or strength envelope 150. Since body model 318 also determines flexibility of other joints, even when not directly measured, even without full sensor instrumentation of individual 102, body model 318 may derive movement and thus flexibility and strength ranges of other joints and/or muscles.

In one embodiment, flex server 310 also includes an envelope display generator 320 that interactively generates a display 330 illustrating at least part of overall motion, flexibility and/or strength envelope 150. In the example of FIG. 3, display 330 shows right hip strength 335 and flexibility resulting from the illustrated bending at the waist of individual 102. In particular, the strength of individual 102 varies for different body positions. For example, arm strength may be reduced as individual 102 reaches out further.

The greater the number of sensors 302 applied to different parts of individual 102, the greater the accuracy of body model 318, and thus the greater the accuracy of overall motion, flexibility and/or strength envelope 150.

Envelope display generator 320 may be interactive to allow a user (e.g., a doctor or individual 102) to selectively view certain types of motion, flexibility and/or strength within display 330. Where overall motion, flexibility and/or strength envelope 150 contains many different types of movement, the user may elect to view one or more of these movements and strengths on display 130.

In the example of FIG. 3, display 330 shows a ‘stick’ FIG. 332 that represents individual 102, a flexibility range 334, and a strength range 335 that represents the maximum range of right-hip motion, flexibility and/or strength achieved by individual 102 for a given weight 303. In one embodiment, memory 312 also stores a recorded motion, flexibility and/or strength envelope 350. In one embodiment, recorded motion, flexibility and/or strength envelope 350 represents average motion, flexibility and/or strength a normal healthy individual of a certain body type and age. In another embodiment, recorded motion, flexibility and/or strength envelope 350 represents a previously recorded overall motion, flexibility and/or strength envelope 150 of individual 102. Based upon recorded motion, flexibility and/or strength envelope 350, generator 320 may concurrently display expected motion, flexibility and/or strength and/or previously achieved motion, flexibility and/or strength of individual 102 as motion, flexibility and/or strength range 336. Thus, the user may compare current motion, flexibility and/or strength of individual 102 to expected or previously recorded motion, flexibility and/or strength envelope 350. For example, an expected overall motion, flexibility and/or strength envelope may be based upon an average of similar individuals, or based upon one or more of a current age, physical condition, injury status, of the individual. For clarity of illustration, the example of FIG. 3 shows a two dimensional motion, flexibility and/or strength range; however, system 300 may display multiple motion, flexibility and/or strength ranges from overall motion, flexibility and/or strength envelope 150 concurrently without departing from the scope hereof. Further, based upon overall motion, flexibility and/or strength envelope 150, system 300 may display motion, flexibility and/or strength ranges corresponding to any joint of individual 102, even when that motion, flexibility and/or strength is not directly measured.

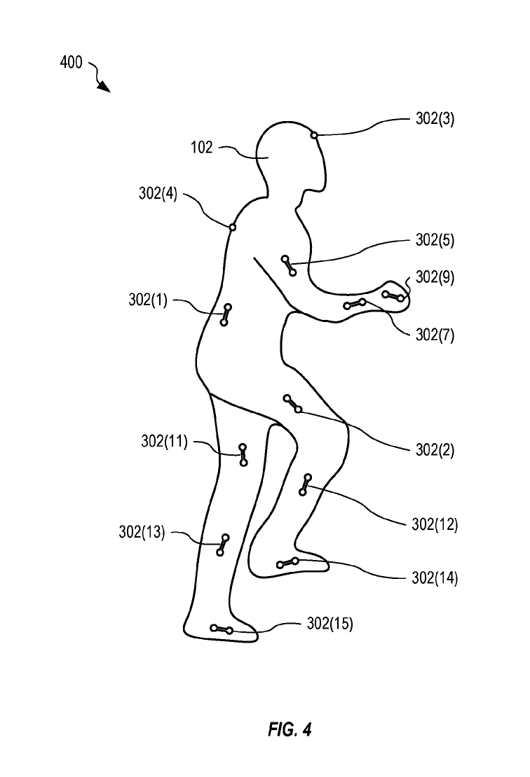

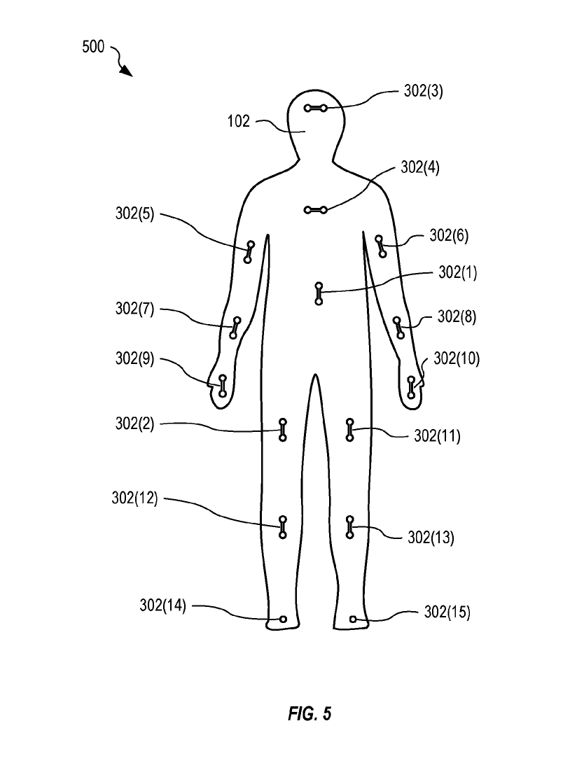

FIG. 4 is a right side view 400 of individual 102 and FIG. 5 is a front view 500 of individual 102, each view 400, 500 illustrating example positioning of sensors 302 on individual 102. FIGS. 4 and 5 are best viewed together with the following description.

Continuing with the example of FIG. 3, sensor 302(1) is positioned at the lower-back area of individual 102 and sensor 302(2) is positioned at a right-thigh area of individual 102. Sensor 302(3) is positioned on the head of individual 102 and measures head movement. In one embodiment, sensor 302(3) is configured with a hat worn by individual 102. Sensor 302(4) is positioned at the upper back (between scapula) of individual 102. Sensor 302(5) is positioned on an upper right arm of individual 102. Sensor 302(6) is positioned on an upper left arm of individual 102. Sensor 302(7) is positioned on a right forearm of individual 102. Sensor 302(8) is positioned on a left forearm of individual 102. Sensor 302(9) is positioned on the back of a right hand of individual 102. Sensor 302(10) is positioned on the back of a left hand of individual 102. Sensor 302(11) is positioned on a left thigh of individual 102. Sensor 302(12) is positioned on a lower right leg of individual 102. Sensor 302(13) is positioned on a lower left leg of individual 102. Sensor 302(14) is positioned on a right foot of individual 102. Sensor 302(15) is positioned on a left foot of individual 102.

More or fewer sensors 302, in the same or different body locations, may be configured with individual 102 without departing from the scope hereof. For example, to measure finger flexibility and strength, gloves may be configured with a plurality of sensors 302 that measure movement and strength of each finger segment. Sensors 302 may also be configured to measure other parameters, such as temperature, heart rate, etc., without departing from the scope hereof.

In one embodiment, each sensor 302(1)-(15) measures motion (linear displacement and rotation) in three perpendicular axes X, Y and Z (often referred to as six axis measurement). Where individual 102 is to be monitored continuously (e.g., for an entire day or longer period), at least some of sensors 302 may be surgically implanted within individual 102. Certain other sensors 302 may be adhesively (e.g., as in a band aid) attached to individual 102. Certain other sensors 302 may be configured with clothing worn by individual 102. Certain sensors 302 may measure other characteristics than motion, such as blood flow, heart rate, oxygen level, or other information. Where individual 102 is periodically but infrequently tested (e.g., certain movements tests once a week), sensors 302 may be configured with test equipment (e.g., exercise equipment). As data is collected, it is transmitted to flex server 310 that operates to create or update overall motion, flexibility and/or strength envelope 150 of individual 102. Sensors 302 may be selected to measure one or more of displacement, velocity, and acceleration. Alternatively these parameters may be derived from other inputs (e.g., video). Regardless of the information source, motion, flexibility and/or strength envelope 150 may take several forms—as a displacement envelope, as a velocity envelope, and as an acceleration envelope.

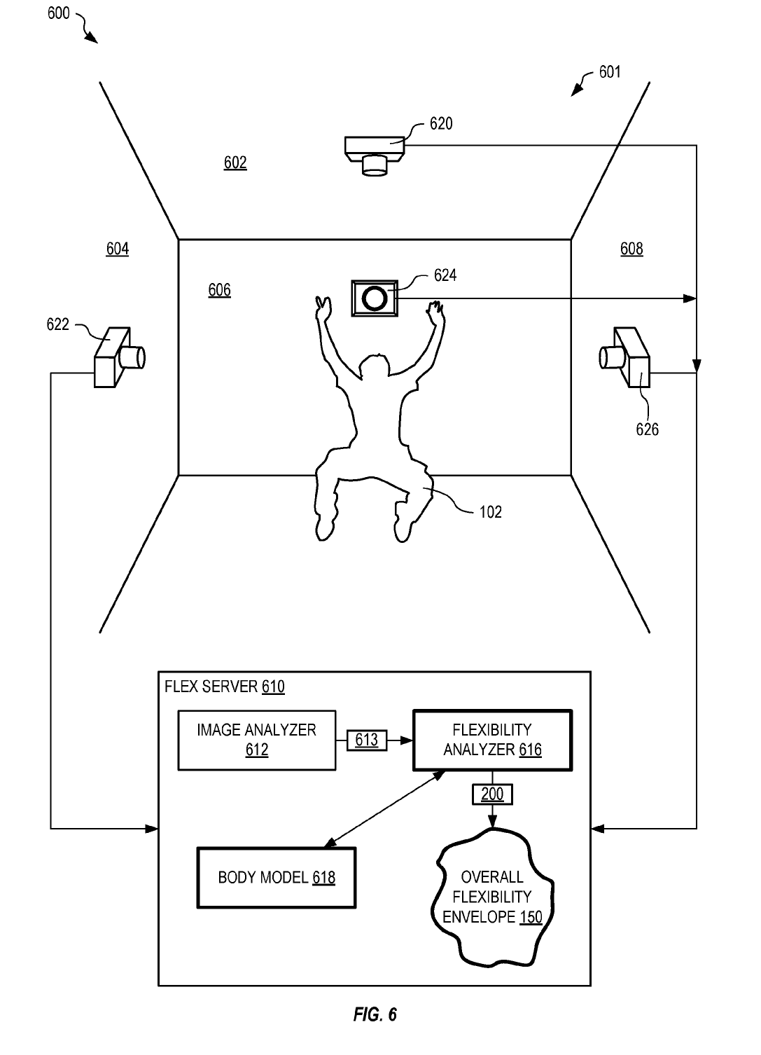

FIG. 6 shows one example system 600 that utilizes flex server 310 of FIG. 3, together with a plurality of cameras 620(1)-(4) mounted to capture images within a room 601, to determine overall motion, flexibility and/or strength envelope 150 of individual 102. Camera 620(1) is configured on a ceiling 602 of room 601, and cameras 620(2)-(4) are mounted on walls 604, 606, and 608, respectively, of room 601. System 600 may have more or fewer cameras 620 without departing from the scope hereof. System 600 includes a flex server 610 that is similar to flex server 310 of FIG. 3, and includes a memory and processor that are not shown for clarity of illustration. Flex server 610 receives images (e.g., a sequence of sequentially captured images) concurrently from each camera 620, and includes an image analyzer 612 that processes these images to determine movements 613 of individual 102 within room 601 and also identify any weights that individual 102 is using. Flex server 610 also includes a motion, flexibility and/or strength analyzer 616 and a body model 618 that are similar to flexibility analyzer 316 and body model 318 of FIG. 3. Flexibility analyzer 616 inputs identified weights, or sensed forces and movement 613 into body model 618 and determines motion, flexibility and/or strength inputs 200 for input to overall motion, flexibility and/or strength envelope 150.

As individual 102 moves within room 601, system 600 uses cameras 620 and image analyzer 612 to process images received from each camera 620 to determine movements 613 and to identify objects carried or moved, as known in the art. In certain embodiments, visual markers are attached to certain points on the body of individual 102 to facilitate movement tracking. In another embodiment, cameras 620 capture infra-red images, wherein system 600 also includes an infrared projector (not shown) that projects a pattern into room 601 that facilitates detection of motion by individual 102.

Other methods of movement and strength capture may be used without departing from the scope hereof. For example, exercise equipment may be configured to measure forces and movement of individual 102 during exercise, wherein the forces and movement information is input to flexibility analyzer 316 for conversion into motion, flexibility and/or strength inputs 200 using body model 318 and then used to form overall motion, flexibility and/or strength envelope 150. In another example, rehabilitation equipment is configured to record strength and movement for input to system 300.

Beyond using sensors affixed motion, gyroscope and accelerometer sensors or external cameras it should be understood that in certain embodiments other means of motion capture—e.g. heat signature from thermal cameras, or sound based signatures from audible or other sonic means or a sonar-like system or a radar-like system or a GPS type device may be utilized to ultimately generate the displacement, velocity or acceleration data for flexibility envelope 150.

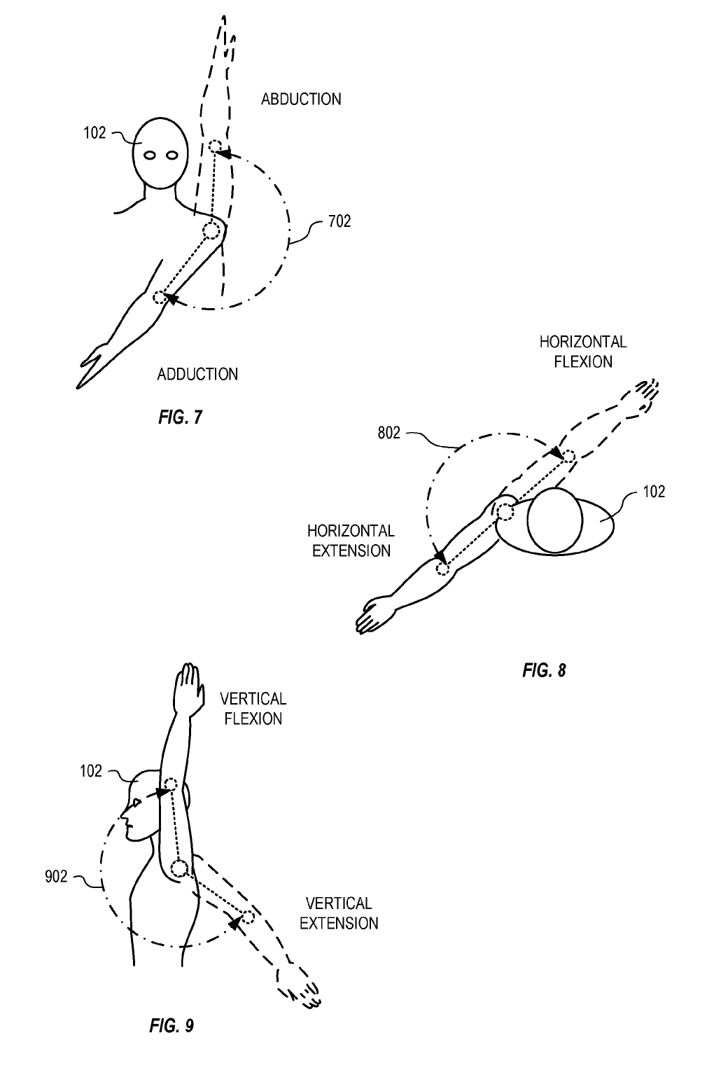

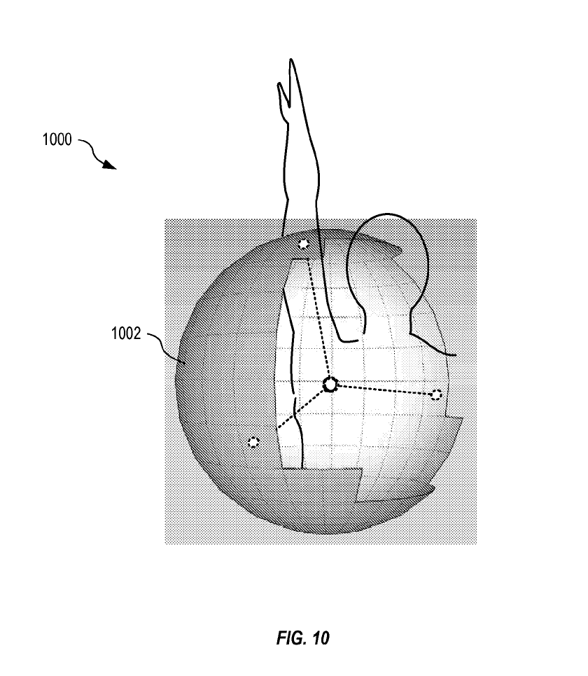

FIGS. 7, 8 and 9 show example movement of the left-shoulder of individual 102. FIG. 7 shows movement of the shoulder from abduction through adduction, giving a first range of motion 702. FIG. 8 shows the shoulder moving from horizontal flexion through a horizontal extension, giving a second range of motion 802. FIG. 9 shows the shoulder moving from vertical extension through vertical flexion, giving a third range of motion 902. FIGS. 7 through 9 thus show conventional evaluation of shoulder movement. Although each range of motion may be measured and evaluated separately, overall motion, flexibility and/or strength envelope 150 encompasses the overall motion, flexibility and/or strength of individual 102, and thereby allows the shoulder flexibility to be viewed as a whole as shown in FIG. 10.

FIG. 10 shows one example rendering 1000 illustrating shoulder motion, flexibility and/or strength generated from overall motion, flexibility and/or strength envelope 150. Rendering 1000 is from a rear perspective of individual 102, where a shell 1002 indicates overall motion, flexibility and/or strength of the left shoulder of individual 102 by representing points where the elbow has reached relative to the torso. For example, as motion, flexibility and/or strength inputs 200 are added to overall motion, flexibility and/or strength envelope 150, this shell is filled based upon the achieved movement (and derived movement/flexibility) within body model 318. Overall motion, flexibility and/or strength envelope 150 may be considered to include such shells for many, if not all, joints of individual 102, thereby allowing flexibility of one or more joints to be easily viewed and assimilated by medical practitioners and the individual.

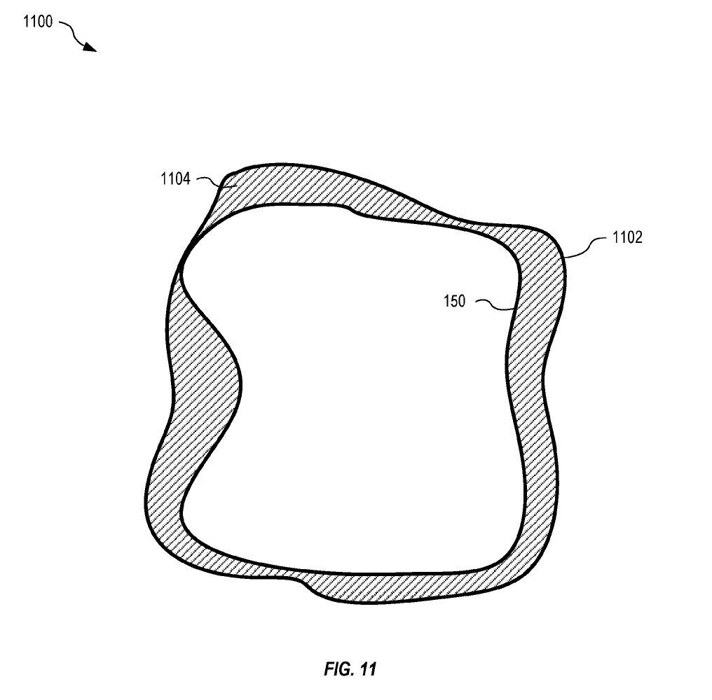

FIG. 11 shows motion, flexibility and/or strength envelope 150 and an exemplary overlapping comfort/discomfort envelope 1102. In addition to generating motion, flexibility and/or strength envelope 150, which is objective and quantitative, certain embodiments hereof may be configured to create overlapping comfort/discomfort envelope 1102. It is useful to understands the subjective and/or physiologic consequence of the flexibility range defined within overall motion, flexibility and/or strength envelope 150 and the coordinate physiologic or subjective feelings of the organism, particularly when moving at extreme ranges of overall motion, flexibility and/or strength envelope 150. Overall motion, flexibility and/or strength envelope 150 is represented as an inside line, and an outside line represents comfort/discomfort envelope 1102. As individual 102 moves at the extremes of overall motion, flexibility and/or strength envelope 150 using the motions of the body and/or each flex element needed to achieve this motion, flexibility and/or strength, individual 102 may subjectively experience more discomfort as they achieve these extremes of overall motion, flexibility and/or strength envelope 150. For example, where no discomfort occurs, comfort/discomfort envelope 1102 may have the same shape and size as overall motion, flexibility and/or strength envelope 150. However, where discomfort occurs, comfort/discomfort envelope 1102 may extend beyond overall motion, flexibility and/or strength envelope 150 to indicate the discomfort, as indicated in FIG. 11 by shaded area 1104. In one embodiment, inputting contemporaneous discomfort or pain information—either verbally with coordinate time synched recording, or physiologic parameters such as increasing heart rate or BP—allows greater granular component information of motion, flexibility and/or strength envelope 150 to be revealed. For example, in a given individual while their quantitative flex/motion envelope might not change between two time points (e.g. one while well trained, another while immobile for a period of time), the quality and the subjective feeling and/or objective physiologic response (i.e. painfulness) as indicated by comfort/discomfort envelope 1102 may vary. Showing the overlap between subjective and physiologic parameter response further enriches the information obtained, and provides both objective as well as subjective information that is useful to assess, counsel, train and/or intervene in and with the organism (e.g., individual 102). System 300 may generate, analyze and display overlapping envelopes and the interaction between these envelopes may be analyzed as well.

In the embodiments described herein, the motion, flex and subjective comfort discomfort envelopes may be determined, stored, displayed and analyzed on a handheld device—e.g. smartphone tablet, smartwatch, phablet, or analogous digital or analog display and analysis means. Similarly, a desktop, laptop, mainframe or other computer system or embedded hardware, firmware system may be utilized. The embodiments described herein may also be embedded into a medical device—e.g. a holter monitor, event monitor, implanted pacemaker or defibrillator, stent, valve, sensor, pump system, orthopedic device, implant or system; metabolic, respiratory, neural, auditory, otic, ophthalmic, gastrointestinal or other physiologic system device, implant or system.

In the embodiments described herein, the data may be telemeter or otherwise sent to a secondary repository, storage or analysis system and/or up to a ‘cloud.” Similarly, said signals and data may be retrieved form the like.

In the embodiments described herein, data may be sent via electromagnetic, radiofrequency, telephonic, optical, thermal, electro-optical, Bluetooth, near field or other transmission means.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween. In particular, the following embodiments are specifically contemplated, as well as any combinations of such embodiments that are compatible with one another:

(A) A method for determining an overall motion, flexibility and/or strength envelope for an individual, including capturing force, movement and position data of the individual, and processing the force, movement and position data to generate an overall motion, flexibility and/or strength envelope for the individual defining overall motion, flexibility and/or strength of the individual.

(B) In the method denoted as (A), the force, movement and position data being captured from at least two sensors configured with the individual.

(C) In either of the methods denoted as (A) and (B), the force, movement and position data being captured from one sensor configured with the individual over time.

(D) In any of the methods denoted as (A)-(C), the sensors being implanted within the individual.

(E) In any of the methods denoted as (A)-(D), the at least two sensors being selected from the group including strain gauges, accelerometers, gyroscopes, displacement sensors, proximity sensors, hall effect sensors, optical encoders, potentiometers, linear and rotary sensors, eddy-current sensors, reflective light sensors, pressure sensors, force sensors, tilt sensors, heart rate monitors, blood flow sensors, oxygen level sensors, and vibration sensors.

(F) In any of the methods denoted as (A)-(E), the force, movement and position data being determined by analyzing imagery of the individual captured by one or more cameras.

(G) In any of the methods denoted as (A)-(F), the step of processing including manipulating a digital model of the individual based upon the force, movement and position data to determine and derive strength, movement and flexibility of at least one joint and/or muscle of the individual.

(H) In any of the methods denoted as (A)-(G), the step of processing the force, movement and position data including comparing a currently determined overall motion, flexibility and/or strength envelope of the individual to a previously determined overall motion, flexibility and/or strength envelope of the individual to identify changes in motion, flexibility and/or strength of the individual.

(I) In any of the methods denoted as (A)-(H), the step of processing the force, movement and position data including comparing a currently determined overall motion, flexibility and/or strength envelope of the individual to an expected overall motion, flexibility and/or strength envelope.

(J) A system for determining an overall motion, flexibility and/or strength envelope for an individual, includes a flexibility server having memory and a processor and adapted to receive force, movement and position data of the individual, and a motion, flexibility and/or strength analyzer, implemented as machine readable instructions stored in the memory and executed by the digital processor, capable of processing the force, movement and position data to generate an overall motion, flexibility and/or strength envelope for the individual defining overall motion, flexibility and/or strength of the individual.

(K) The system denoted above as (J), further including at least one sensor configured for sensing and generating the force, movement and position data.

(L) In either of the systems denoted as (J) and (K), the sensors including one or more of implantable sensors implantable in the individual to sense the force, movement and position data from the individual's body, wearable sensors that attach to the individual's body to sense the force, movement and position data, and off the body sensors for sensing the force, movement and position data from the individual.

(M) In any of the systems denoted as (J)-(L), the off body sensors including one or both of a machine for sensing the individual's force, movement and position, and at least two cameras for sensing the individual's force, movement and position.

(N) Any of the systems denoted as (J)-(M), further including an image analyzer, implemented as machine readable instructions stored in the memory and executed by the digital processor, capable of processing images from the at least two cameras to determine the force, movement and position data.

(O) Any of the systems denoted as (J)-(M), further including a relay device for receiving the force, movement and position data from the sensor and for sending the force, movement and position data to the flexibility analyzer.

(P) Any of the systems denoted as (J)-(L), further including an envelope display generator, implemented as machine readable instructions stored in the memory and executed by the digital processor, capable of interactively providing a view of at least part of the overall motion, flexibility and/or strength envelope.

(Q) In any of the systems denoted as (J)-(P), the envelope display generator being further capable of comparing and displaying a difference between the overall motion, flexibility and/or strength envelope and a recorded motion, flexibility and/or strength envelope.

(R) Any of the systems denoted as (J)-(Q), further including a body model implemented within the memory and operable to model one or more of force, size, weight, and movement of the individual.

(S) Any of the systems denoted as (J)-(R), further including an output device for prompting the individual to move at least part of the individual through a range of suggested or designated motions.

(T) In any of the systems denoted as (J)-(L), said motions being performed to the limit of acceptable discomfort or capability.

(U) A system for determining an overall motion envelope for an individual, including means for capturing force and motion of the individual, means for recording and analyzing said force and motion data, means for storing raw and processed information, means for converting the force and motion data into a motion, flexibility and/or strength envelope, means for displaying the motion, flexibility and/or strength envelope, and means for comparing the motion, flexibility and/or strength envelope at differing time points and under differing conditions.

(V) In the system denoted as (U), the motion being determined via flexibility of one or more of: the individual overall, a component of the individual, and a flex element of the individual.

(W) Either of the systems denoted as (U) and (V), further including means for capturing both objective motion and objective physiologic response to the motion.

(X) In any of the systems denoted as (U)-(W), the objective physiologic response comprising one or more of heart rate, blood pressure, degree of sweating, heart rate variability, blood pressure variability, catechol levels, and other markers of stress.

(Y) In any of the systems denoted as (U)-(X), the objective physiologic response allowing contemporaneous capture, analysis and generation of a comfort/discomfort/physiologic response/consequence envelope overlapping the motion/flexibility envelope.

(Z) In any of the systems denoted as (U)-(W), the objective physiologic response allowing the determination of the consequences of these interactions.

(AA) In any of the systems denoted as (U)-(Z), contemporaneous sound or physiologic data being inputted to allow overlay and/or contemporaneous time-synched analysis of said data to determine pain or discomfort envelope or contours overlaying the motion/flex envelope.

(AB) Any of the systems denoted as (U)-(AA), further including input sensors that are applied, implanted or otherwise affixed position sensors, including accelerometers, gyroscopes strain gauges.

(AC) In any of the systems denoted as (U)-(AB), the input sensors being capable of sensing one or more of image, sound, heat, vibration, odor/vapors, and chemical markers detectors.

(AD) In any of the systems denoted as (U)-(AC), data from the input sensors being used to determine the force, motion and flexibility.

(AE) In any of the systems denoted as (U)-(AD), the flexibility envelope being based upon one or more of motion (dimension), velocity, and acceleration data.

(AF) Any of the systems denoted as (U)-(AE), further including means for portraying the flexibility envelope in one or more of a 2D, a 3D and a 4D representation.

(AG) In any of the systems denoted as (U)-(AF), the motion, flexibility and/or strength envelope being captured from force and motion that is either active/volition or stimulated.

(AH) In any of the systems denoted as (U)-(AG), the motion, flexibility and/or strength envelope is captured from force and motion that is passive and/or done by third party or device.

(AI) In any of the systems denoted as (U)-(AH), the motion, flexibility and/or strength envelope being captured from flexibility that is the same, greater or less than the motion envelope.

(AJ) Any of the systems denoted as (U)-(AI), further including capturing a comfort/discomfort envelope that is subjective.

(AK) A method for determining an overall motion, flexibility and/or strength envelope for an individual, includes the steps of capturing force, movement and position data from at least one sensor or motion detection means configured with the individual, analyzing the force, movement and position data to generate the overall motion, flexibility and/or strength envelope for the individual, and generating a display from the overall motion, flexibility and/or strength envelope to show motion, flexibility and/or strength of the individual.

(AL) The method denoted as (AK), further including comparing comparative states of the flexibility envelope over time.

(AM) Either method denoted as (AK) and (AL), further including prompting the individual to perform a program of motions, exercises or suggested movements to define whole body or regional motion, flexibility, strength and subjective difficulty envelopes to provide standardization for comparison.

(AN) A software product having instructions, stored on non-transitory computer-readable media, wherein the instructions, when executed by a computer, perform steps for determining an overall motion, flexibility and/or strength envelope for an individual, including instructions for capturing force and movement data, instructions for analyzing the force and movement data, instructions for converting the force and movement data into a graphical representation of movement in either 1, 2, 3 or 4D, and instructions for comparing movement of any aspect of full or partial motion, flexibility and/or strength envelope to either another point of time of the envelope of the same individual or of a database of performance.

(AO) The software product denoted as (AN), further including instructions for determining both quantitatively and graphically the comparative states of the motion, flexibility and/or strength envelope and of the delta (change).

(AP) A device for portraying, comparing and displaying the overall motion, flexibility and/or strength envelope.

(AQ) A system to allow data to be sent to the cloud, be security encrypted and then downloaded by appropriate, security cleared user.

(AR) A series of instructions, exercises, directives to attempt to regain the motion/flexibility envelope if a decline has occurred, using any of the systems, methods, and software products denoted as (A)-(AQ) to monitor progress and guide therapy.

(AS) In any of the systems, methods, and software products denoted as (A)-(AR), the systems, methods, software products, and devices being embedded into a medical device selected from the group including a holter monitor, event monitor, implanted pacemaker or defibrillator, stent, valve, sensor, pump system, orthopedic device, implant or system; metabolic, respiratory, neural, auditory, otic, ophthalmic, gastrointestinal or other physiologic system device, implant or system.

(AT) In any of the systems, methods, and software products denoted as (A)-(AS), the data being telemeter or otherwise sent to a secondary repository, storage or analysis system and/or up to a ‘cloud.”

(AU) In any of the systems, methods, and software products denoted as (A)-(AT), the data is received from a secondary repository, storage or analysis system and/or up to a ‘cloud.”

(AV) In any of the systems, methods, and software products denoted as (A)-(AR), the data being sent via electromagnetic, radiofrequency, telephonic, optical, thermal, electro-optical, Bluetooth, near field or other transmission means.

What is claimed is:

1. A method for determining an overall motion, flexibility and/or strength envelope for an individual, comprising the steps of:
capturing force, movement and position data of the individual; and
processing the force, movement and position data to generate an overall motion, flexibility and/or strength envelope for the individual defining overall motion, flexibility and/or strength of the individual.

2. The method of claim 1, wherein the force, movement and position data is captured from at least two sensors configured with the individual.

3. The method of claim 2, wherein the force, movement and position data is captured from one sensor configured with the individual over time.

4. The method of claim 3, wherein the sensors are implanted within the individual.

5. The method of claim 4, the at least two sensors being selected from the group including strain gauges, accelerometers, gyroscopes, displacement sensors, proximity sensors, hall effect sensors, optical encoders, potentiometers, linear and rotary sensors, eddy-current sensors, reflective light sensors, pressure sensors, force sensors, tilt sensors, heart rate monitors, blood flow sensors, oxygen level sensors, and vibration sensors.

6. The method of claim 5, wherein the force, movement and position data is determined by analyzing imagery of the individual captured by one or more cameras.

7. The method of claim 6, the step of processing comprising manipulating a digital model of the individual based upon the force, movement and position data to determine and derive strength, movement and flexibility of at least one joint and/or muscle of the individual.

8. The method of claim 7, the step of processing the force, movement and position data comprising comparing a currently determined overall motion, flexibility and/or strength envelope of the individual to a previously determined overall motion, flexibility and/or strength envelope of the individual to identify changes in motion, flexibility and/or strength of the individual.

9. The method of claim 8, the step of processing the force, movement and position data comprising comparing a currently determined overall motion, flexibility and/or strength envelope of the individual to an expected overall motion, flexibility and/or strength envelope.

10. A system for determining an overall motion, flexibility and/or strength envelope for an individual, comprising:
a flexibility server having memory and a processor and adapted to receive force, movement and position data of the individual; and
a motion, flexibility and/or strength analyzer, implemented as machine readable instructions stored in the memory and executed by the digital processor, capable of:
processing the force, movement and position data to generate an overall motion, flexibility and/or strength envelope for the individual defining overall motion, flexibility and/or strength of the individual.

11. The system of claim 10, further comprising at least one sensor configured for sensing and generating the force, movement and position data.

12. The system of claim 11, the sensors comprising one or more of implantable sensors implantable in the individual to sense the force, movement and position data from the individual's body, wearable sensors that attach to the individual's body to sense the force, movement and position data, and off the body sensors for sensing the force, movement and position data from the individual.

13. The system of claim 12, the off body sensors comprising one or both of a machine for sensing the individual's force, movement and position, and at least two cameras for sensing the individual's force, movement and position.

14. The system of claim 13, further comprising an image analyzer, implemented as machine readable instructions stored in the memory and executed by the digital processor, capable of processing images from the at least two cameras to determine the force, movement and position data.

15. The system of claim 14, further comprising a relay device for receiving the force, movement and position data from the sensor and for sending the force, movement and position data to the flexibility analyzer.

16. The system of claim 15, further comprising an envelope display generator, implemented as machine readable instructions stored in the memory and executed by the digital processor, capable of interactively providing a view of at least part of the overall motion, flexibility and/or strength envelope.

17. The system of claim 16, the envelope display generator further capable of comparing and displaying a difference between the overall motion, flexibility and/or strength envelope and a recorded motion, flexibility and/or strength envelope.

18. The system of claim 17, further comprising a body model implemented within the memory and operable to model one or more of force, size, weight, and movement of the individual.

19. The system of claim 18, further comprising an output device for prompting the individual to move at least part of the individual through a range of suggested or designated motions.

20. The system of claim 19, wherein said motions are performed to the limit of acceptable discomfort or capability.

21. A system for determining an overall motion envelope for an individual, comprising:
means for capturing force and motion of the individual;
means for recording and analyzing said force and motion data;
means for storing raw and processed information;
means for converting the force and motion data into a motion, flexibility and/or strength envelope;
means for displaying the motion, flexibility and/or strength envelope; and
means for comparing the motion, flexibility and/or strength envelope at differing time points and under differing conditions.

22. The system of claim 21, wherein the motion is determined via flexibility of one or more of: the individual overall, a component of the individual, and a flex element of the individual.

23. The system of claim 22, further comprising means for capturing both objective motion and objective physiologic response to the motion.

24. The system of claim 23, the objective physiologic response comprising one or more of heart rate, blood pressure, degree of sweating, heart rate variability, blood pressure variability, catechol levels, and other markers of stress.

25. The system of claim 24, the objective physiologic response allowing contemporaneous capture, analysis and generation of a comfort/discomfort/physiologic response/consequence envelope overlapping the motion/flexibility envelope.

26. The system of claim 25, the objective physiologic response allowing the determination of the consequences of these interactions.

27. The system of claim 26, wherein contemporaneous sound or physiologic data is inputted to allow overlay and/or contemporaneous time-synched analysis of said data to determine pain or discomfort envelope or contours overlaying the motion/flex envelope.

28. The system of claim 27, further comprising input sensors that are applied, implanted or otherwise affixed position sensors, including accelerometers, gyroscopes strain gauges.

29. The system of claim 28, the input sensors being capable of sensing one or more of image, sound, heat, vibration, odor/vapors, and chemical markers detectors.

30. The system of claim 29, wherein data from the input sensors are used to determine the force, motion and flexibility.

31. The system of claim 30, the flexibility envelope based upon one or more of motion (dimension), velocity, and acceleration data.

32. The system of claim 31, further comprising means for portraying the flexibility envelope in one or more of a 2D, a 3D and a 4D representation.

33. The system of claim 32, wherein the motion, flexibility and/or strength envelope is captured from force and motion that is either active/volition or stimulated.

34. The system of claim 33, wherein the motion, flexibility and/or strength envelope is captured from force and motion that is passive and/or done by third party or device.

35. The system of claim 34, wherein the motion, flexibility and/or strength envelope is captured from flexibility that is the same, greater or less than the motion envelope.

36. The system of claim 35, further comprising means for capturing a comfort/discomfort envelope that is subjective.

37. A method for determining an overall motion, flexibility and/or strength envelope for an individual, comprising the steps of:
capturing force, movement and position data from at least one sensor or motion detection means configured with the individual;
analyzing the force, movement and position data to generate the overall motion, flexibility and/or strength envelope for the individual; and
generating a display from the overall motion, flexibility and/or strength envelope to show motion, flexibility and/or strength of the individual.

38. The method of claim 37, further comprising comparing comparative states of the flexibility envelope over time.

39. The method of claim 38, further comprising prompting the individual to perform a program of motions, exercises or suggested movements to define whole body or regional motion, flexibility, strength and subjective difficulty envelopes to provide standardization for comparison.

40. A software product comprising instructions, stored on non-transitory computer-readable media, wherein the instructions, when executed by a computer, perform steps for determining an overall motion, flexibility and/or strength envelope for an individual, comprising:
instructions for capturing force and movement data;
instructions for analyzing the force and movement data;
instructions for converting the force and movement data into a graphical representation of movement in either 1, 2, 3 or 4D; and
instructions for comparing movement of any aspect of full or partial motion, flexibility and/or strength envelope to either another point of time of the envelope of the same individual or of a database of performance.

41. The software product of claim 40, further comprising instructions for determining both quantitatively and graphically the comparative states of the motion, flexibility and/or strength envelope and of the delta (change).

42. A device for portraying, comparing and displaying the overall motion, flexibility and/or strength envelope.

43. A system to allow data to be sent to the cloud, be security encrypted and then downloaded by appropriate, security cleared user.

44. A series of instructions, exercises, directives to attempt to regain the motion flexibility envelope if a decline has occurred, using the system in claim 21 to monitor progress and guide therapy.

45. Any of the systems and methods of claims 1 through 44, wherein the systems, methods and devices are embedded into a medical device selected from the group including a holter monitor, event monitor, implanted pacemaker or defibrillator, stent, valve, sensor, pump system, orthopedic device, implant or system; metabolic, respiratory, neural, auditory, otic, ophthalmic, gastrointestinal or other physiologic system device, implant or system.

46. Any of the systems and methods of claims 1 through 44, wherein the data is telemeter or otherwise sent to a secondary repository, storage or analysis system and/or up to a ‘cloud.”

47. Any of the systems and methods of claims 1 through 44, wherein the data is received from a secondary repository, storage or analysis system and/or up to a ‘cloud.”

48. Any of the systems and methods of claims 1 through 44, wherein data is sent via electromagnetic, radiofrequency, telephonic, optical, thermal, electro-optical, Bluetooth, near field or other transmission means.
